SEQUENCE LISTING (22 sequences)

SEQ ID NO: 1 - DNA, 257 bp, Artificial sequence
  L5 promoter
ctagaggaaacagctatgaccatgattacgccaagcttgcatgcctgcagctagggcaccaatttgcgattagggcttgacagccacccggccagtagtgcattcttgtgtcaccgcagcagcaaggcggtaggcggatccgagaggatcgtgccggtgccggtgaaaatccggcggcaagattctccggtttgacagccacccggttatcgggtaagctgcaagcatcaccaacttggacgggaaagggagatcgc

SEQ ID NO: 2 - DNA, 1367 bp, Artificial sequence
  OriM
gtggggtggcccctcagcgaaatatctgacttggagctcgtgtcggaccatacaccggtgattaatcgtggtttattatcaagcgtgagccacgtcgccgacgaatttgagcagctctggctgccgtactggtccctggcaagcgacgatctgctcgaggggatctaccgccaaagccgcgcgtcggccctaggccgccggtacatcgaggcgaacccaacagcgctggcaaacctgctggtcgtggacgtagaccatccagacgcagcgctccgagcgctcagcgcccgggggtcccatccgctgcccaacgcgatcgtgggcaatcgcgccaacggccacgcacacgcagtgtgggcactcaacgcccctgttccacgcaccgaatacgcgcggcgtaagccgctcgcatacatggcggcgtgcgccgaaggccttcggcgcgccgtcgatggcgaccgcagttactcaggcctcatgaccaaaaaccccggccacatcgcctgggaaacggaatggctccactcagatctctacacactcagccacatcgaggccgagctcggcgcgaacatgccaccgccgcgctggcgtcagcagaccacgtacaaagcggctccgacgccgctagggcggaattgcgcactgttcgattccgtcaggttgtgggcctatcttcccgccctcatgcggatctacctgccgacccggaacgtggacggactcggccgcgcgatctatgccgagtgccacgcgcgaaacgccgaatttccgtgcaacgacgtgtgtcccggaccgctaccggacagcgaggtccgcgccatcgccaacagcatttggcgttggatcacaaccaagtcgcgcatttgggcggacgggatcgtggtctacgaggccacactcagtgcgcgccatgcggccatctcgcggaagggcgcagcagcgcgcacggcggcgagcacagttgcgcggcgcgcaaagtccgcgtcagccatggaggcattgctatgagcgacggctacagcgacggctacagcgacggctacaactggcagccgactgtccgcaaaaagcggcgcgtgaccgccgccgaaggcgctcgaatcaccggactatccgaacgccacgtcgtccggctcgtggcgcaggaacgcagcgagtggttcgccgagcaggctgcacgccgcgaacgcatccgcgcctatcacgacgacgagggccactcttggccgcaaacggccaaacatttcgggctgcatctggacaccgttaagcgactcggctatcgggcgaggaaagagcgtgcggcagaacaggaagcggctcaaaaggcccacaacgaagccgacaatccaccgctgttctaa

SEQ ID NO: 3 - DNA, 1185 bp, Artificial sequence
  mNeon_green-nLuc
atggtgtccaagggcgaagaggacaacatggccagcctgcctgccacccacgagctgcacatcttcggcagcatcaacggcgtggacttcgacatggtgggacagggcaccggcaaccccaacgacggctacgaggaactgaacctgaagtccacaaagggcgacctgcagttcagcccctggattctggtgccccacatcggctacggcttccaccagtacctgccctaccccgacggcatgagccctttccaggccgctatggtggatggcagcggctaccaggtgcaccggaccatgcagtttgaggacggcgccagcctgaccgtgaactaccggtacacatacgagggcagccacatcaagggcgaggcccaagtgaagggcacaggctttccagccgacggccccgtgatgaccaatagcctgacagccgccgactggtgcagaagcaagaaaacctaccccaatgacaagaccatcatcagcaccttcaagtggtcctacaccaccggcaatggcaagcggtacagaagcaccgcccggaccacctacaccttcgccaaacctatggccgccaactacctgaagaaccagcctatgtacgtgttccgcaagaccgagctgaagcactccaagacagaactgaacttcaaagagtggcagaaagccttcaccgggtttgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaa

SEQ ID NO: 4 - protein, 394 aa, Artificial sequence
  mNeon_green-nLuc
MVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTGFEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 5 - DNA, 1200 bp, Artificial sequence
  mTurquoise-nLuc
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgtcctggggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactactttagcgacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggttgcatacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaa

SEQ ID NO: 6 - protein, 399 aa, Artificial sequence
  mTurquoise-nLuc
MVSKGEELFTGVVPILVELDGDVNGHKFSVSGEGEGDATYGKLTLKFICTTGKLPVPWPTLVTTLSWGVQCFARYPDHMKQHDFFKSAMPEGYVQERTIFFKDDGNYKTRAEVKFEGDTLVNRIELKGIDFKEDGNILGHKLEYNYFSDNVYITADKQKNGIKANFKIRHNIEDGGVQLADHYQQNTPIGDGPVLLPDNHYLSTQSKLSKDPNEKRDHMVLLEFVTAAGLHTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 7 - DNA, 1875 bp, Artificial sequence
  mNeon_green-nLuc-mNeon_green
atggtgtccaagggcgaagaggacaacatggccagcctgcctgccacccacgagctgcacatcttcggcagcatcaacggcgtggacttcgacatggtgggacagggcaccggcaaccccaacgacggctacgaggaactgaacctgaagtccacaaagggcgacctgcagttcagcccctggattctggtgccccacatcggctacggcttccaccagtacctgccctaccccgacggcatgagccctttccaggccgctatggtggatggcagcggctaccaggtgcaccggaccatgcagtttgaggacggcgccagcctgaccgtgaactaccggtacacatacgagggcagccacatcaagggcgaggcccaagtgaagggcacaggctttccagccgacggccccgtgatgaccaatagcctgacagccgccgactggtgcagaagcaagaaaacctaccccaatgacaagaccatcatcagcaccttcaagtggtcctacaccaccggcaatggcaagcggtacagaagcaccgcccggaccacctacaccttcgccaaacctatggccgccaactacctgaagaaccagcctatgtacgtgttccgcaagaccgagctgaagcactccaagacagaactgaacttcaaagagtggcagaaagccttcaccgggtttgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgggtggcggttctggtgtgtccaagggcgaagaggacaacatggccagcctgcctgccacccacgagctgcacatcttcggcagcatcaacggcgtggacttcgacatggtgggacagggcaccggcaaccccaacgacggctacgaggaactgaacctgaagtccacaaagggcgacctgcagttcagcccctggattctggtgccccacatcggctacggcttccaccagtacctgccctaccccgacggcatgagccctttccaggccgctatggtggatggcagcggctaccaggtgcaccggaccatgcagtttgaggacggcgccagcctgaccgtgaactaccggtacacatacgagggcagccacatcaagggcgaggcccaagtgaagggcacaggctttccagccgacggccccgtgatgaccaatagcctgacagccgccgactggtgcagaagcaagaaaacctaccccaatgacaagaccatcatcagcaccttcaagtggtcctacaccaccggcaatggcaagcggtacagaagcaccgcccggaccacctacaccttcgccaaacctatggccgccaactacctgaagaaccagcctatgtacgtgttccgcaagaccgagctgaagcactccaagacagaactgaacttcaaagagtggcagaaagccttcacctaa

SEQ ID NO: 8 - protein, 624 aa, Artificial sequence
  mNeon_green-nLuc-mNeon_green
MVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTGFEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGGGSGVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFT

SEQ ID NO: 9 - DNA, 1884 bp, Artificial sequence
  mNeon_green-nLuc-mTurquoise
atggtgtccaagggcgaagaggacaacatggccagcctgcctgccacccacgagctgcacatcttcggcagcatcaacggcgtggacttcgacatggtgggacagggcaccggcaaccccaacgacggctacgaggaactgaacctgaagtccacaaagggcgacctgcagttcagcccctggattctggtgccccacatcggctacggcttccaccagtacctgccctaccccgacggcatgagccctttccaggccgctatggtggatggcagcggctaccaggtgcaccggaccatgcagtttgaggacggcgccagcctgaccgtgaactaccggtacacatacgagggcagccacatcaagggcgaggcccaagtgaagggcacaggctttccagccgacggccccgtgatgaccaatagcctgacagccgccgactggtgcagaagcaagaaaacctaccccaatgacaagaccatcatcagcaccttcaagtggtcctacaccaccggcaatggcaagcggtacagaagcaccgcccggaccacctacaccttcgccaaacctatggccgccaactacctgaagaaccagcctatgtacgtgttccgcaagaccgagctgaagcactccaagacagaactgaacttcaaagagtggcagaaagccttcaccgggtttgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgggtggcggttctggtgtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgtcctggggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactactttagcgacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggtaa

SEQ ID NO: 10 - protein, 627 aa, Artificial sequence
  mNeon_green-nLuc-mTurquoise
MVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFTGFEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGGGSGVSKGEELFTGVVPILVELDGDVNGHKFSVSGEGEGDATYGKLTLKFICTTGKLPVPWPTLVTTLSWGVQCFARYPDHMKQHDFFKSAMPEGYVQERTIFFKDDGNYKTRAEVKFEGDTLVNRIELKGIDFKEDGNILGHKLEYNYFSDNVYITADKQKNGIKANFKIRHNIEDGGVQLADHYQQNTPIGDGPVLLPDNHYLSTQSKLSKDPNEKRDHMVLLEFVTAAG

SEQ ID NO: 11 - DNA, 1893 bp, Artificial sequence
  mTurquoise-nLuc-mNeon_green
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgtcctggggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactactttagcgacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggttgcatacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgggtggcggttctggtatggtgtccaagggcgaagaggacaacatggccagcctgcctgccacccacgagctgcacatcttcggcagcatcaacggcgtggacttcgacatggtgggacagggcaccggcaaccccaacgacggctacgaggaactgaacctgaagtccacaaagggcgacctgcagttcagcccctggattctggtgccccacatcggctacggcttccaccagtacctgccctaccccgacggcatgagccctttccaggccgctatggtggatggcagcggctaccaggtgcaccggaccatgcagtttgaggacggcgccagcctgaccgtgaactaccggtacacatacgagggcagccacatcaagggcgaggcccaagtgaagggcacaggctttccagccgacggccccgtgatgaccaatagcctgacagccgccgactggtgcagaagcaagaaaacctaccccaatgacaagaccatcatcagcaccttcaagtggtcctacaccaccggcaatggcaagcggtacagaagcaccgcccggaccacctacaccttcgccaaacctatggccgccaactacctgaagaaccagcctatgtacgtgttccgcaagaccgagctgaagcactccaagacagaactgaacttcaaagagtggcagaaagccttcacctaa

SEQ ID NO: 12 - protein, 630 aa, Artificial sequence
  mTurquoise-nLuc-mNeon_green
MVSKGEELFTGVVPILVELDGDVNGHKFSVSGEGEGDATYGKLTLKFICTTGKLPVPWPTLVTTLSWGVQCFARYPDHMKQHDFFKSAMPEGYVQERTIFFKDDGNYKTRAEVKFEGDTLVNRIELKGIDFKEDGNILGHKLEYNYFSDNVYITADKQKNGIKANFKIRHNIEDGGVQLADHYQQNTPIGDGPVLLPDNHYLSTQSKLSKDPNEKRDHMVLLEFVTAAGLHTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAGGGSGMVSKGEEDNMASLPATHELHIFGSINGVDFDMVGQGTGNPNDGYEELNLKSTKGDLQFSPWILVPHIGYGFHQYLPYPDGMSPFQAAMVDGSGYQVHRTMQFEDGASLTVNYRYTYEGSHIKGEAQVKGTGFPADGPVMTNSLTAADWCRSKKTYPNDKTIISTFKWSYTTGNGKRYRSTARTTYTFAKPMAANYLKNQPMYVFRKTELKHSKTELNFKEWQKAFT

SEQ ID NO: 13 - DNA, 1899 bp, Artificial sequence
  mTurquoise-nLuc-mTurquoise
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgtcctggggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactactttagcgacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggttgcatacactcgaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgtcctggggcgtgcagtgcttcgcccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactactttagcgacaacgtctatatcaccgccgacaagcagaagaacggcatcaaggccaacttcaagatccgccacaacatcgaggacggcggcgtgcagctcgccgaccactaccagcagaacacccccatcggcgacggccccgtgctgctgcccgacaaccactacctgagcacccagtccaagctgagcaaagaccccaacgagaagcgcgatcacatggtcctgctggagttcgtgaccgccgccgggggtggcggtggttcttaa

SEQ ID NO: 14 - protein, 632 aa, Artificial sequence
  mTurquoise-nLuc-mTurquoise
MVSKGEELFTGVVPILVELDGDVNGHKFSVSGEGEGDATYGKLTLKFICTTGKLPVPWPTLVTTLSWGVQCFARYPDHMKQHDFFKSAMPEGYVQERTIFFKDDGNYKTRAEVKFEGDTLVNRIELKGIDFKEDGNILGHKLEYNYFSDNVYITADKQKNGIKANFKIRHNIEDGGVQLADHYQQNTPIGDGPVLLPDNHYLSTQSKLSKDPNEKRDHMVLLEFVTAAGLHTLEDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILAVSKGEELFTGVVPILVELDGDVNGHKFSVSGEGEGDATYGKLTLKFICTTGKLPVPWPTLVTTLSWGVQCFARYPDHMKQHDFFKSAMPEGYVQERTIFFKDDGNYKTRAEVKFEGDTLVNRIELKGIDFKEDGNILGHKLEYNYFSDNVYITADKQKNGIKANFKIRHNIEDGGVQLADHYQQNTPIGDGPVLLPDNHYLSTQSKLSKDPNEKRDHMVLLEFVTAAGGGGGS

SEQ ID NO: 15 - DNA, 1884 bp, Artificial sequence
  Antares2
atggtgagcaagggcgaggagctgatcaaggagaacatgagaagcaagctgtacctggaaggcagcgtgaacggccaccagttcaagtgcacccacgaaggggagggcaagccctacgagggcaagcagaccaacaggatcaaggtggtggagggaggccccctgccgttcgcattcgacatcctggccacccactttatgtacgggagcaaggtgttcatcaagtaccccgccgacctccccgattattttaagcagtccttccctgagggcttcacatgggagagagtcatggtgttcgaagacgggggcgtgctgaccgccacccaggacaccagcctccaggacggcgagctcatctacaacgtcaaggtcagaggggtgaacttcccagccaacggccccgtgatgcagaagaaaacactgggctgggagcccagcaccgagaccatgtaccccgctgacggcggcctggaaggcagatgcgacaaggccctgaagctcgtgggcgggggccacctgcacgtcaacttcaagaccacatacaagtccaagaaacccgtgaagatgcccggcgtccactacgtggaccgcagactggaaagaatcaaggaggccgacaacgagacctacgtcgagcagtacgagcacgctgtggccagatactccaacctgggcggaggcttcacactcgaagatttcgttggggactggcgacagacagccggctacaacctgagtcaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggataatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcgtctgcatgaacgcattctggcgaggcacgagctgatcaaggagaacatgagaagcaagctgtacctggaaggcagcgtgaacggccaccagttcaagtgcacccacgaaggggagggcaagccctacgagggcaagcagaccaacaggatcaaggtggtggagggaggccccctgccgttcgcattcgacatcctggccacccactttatgtacgggagcaaggtgttcatcaagtaccccgccgacctccccgattattttaagcagtccttccctgagggcttcacatgggagagagtcatggtgttcgaagacgggggcgtgctgaccgccacccaggacaccagcctccaggacggcgagctcatctacaacgtcaaggtcagaggggtgaacttcccagccaacggccccgtgatgcagaagaaaacactgggctgggagcccagcaccgagaccatgtaccccgctgacggcggcctggaaggcagatgcgacaaggccctgaagctcgtgggcgggggccacctgcacgtcaacttcaagaccacatacaagtccaagaaacccgtgaagatgcccggcgtccactacgtggaccgcagactggaaagaatcaaggaggccgacaacgagacctacgtcgagcagtacgagcacgctgtggccagatactccaacctgggcggaggcatggacgagctgtacaagtaa

SEQ ID NO: 16 - protein, 627 aa, Artificial sequence
  Antares2
MVSKGEELIKENMRSKLYLEGSVNGHQFKCTHEGEGKPYEGKQTNRIKVVEGGPLPFAFDILATHFMYGSKVFIKYPADLPDYFKQSFPEGFTWERVMVFEDGGVLTATQDTSLQDGELIYNVKVRGVNFPANGPVMQKKTLGWEPSTETMYPADGGLEGRCDKALKLVGGGHLHVNFKTTYKSKKPVKMPGVHYVDRRLERIKEADNETYVEQYEHAVARYSNLGGGFTLEDFVGDWRQTAGYNLSQVLEQGGVSSLFQNLGVSVTPIQRIVLSGENGLKIDIHVIIPYEGLSGDQMGQIEKIFKVVYPVDNHHFKVILHYGTLVIDGVTPNMIDYFGRPYEGIAVFDGKKITVTGTLWNGNKIIDERLINPDGSLLFRVTINGVTGWRLHERILARHELIKENMRSKLYLEGSVNGHQFKCTHEGEGKPYEGKQTNRIKVVEGGPLPFAFDILATHFMYGSKVFIKYPADLPDYFKQSFPEGFTWERVMVFEDGGVLTATQDTSLQDGELIYNVKVRGVNFPANGPVMQKKTLGWEPSTETMYPADGGLEGRCDKALKLVGGGHLHVNFKTTYKSKKPVKMPGVHYVDRRLERIKEADNETYVEQYEHAVARYSNLGGGMDELYK

SEQ ID NO: 17 - DNA, 692 bp, Artificial sequence
  Antares
tggtgagcaagggcgaggagctgatcaaggagaacatgagaagcaagctgtacctggaaggcagcgtgaacggccaccagttcaagtgcacccacgaaggggagggcaagccctacgagggcaagcagaccaacaggatcaaggtggtggagggaggccccctgccgttcgcattcgacatcctggccacccactttatgtacgggagcaaggtgttcatcaagtaccccgccgacctccccgattattttaagcagtccttccctgagggcttcacatgggagagagtcatggtgttcgaagacgggggcgtgctgaccgccacccaggacaccagcctccaggacggcgagctcatctacaacgtcaaggtcagaggggtgaacttcccagccaacggccccgtgatgcagaagaaaacactgggctgggagcccagcaccgagaccatgtaccccgctgacggcggcctggaaggcagatgcgacaaggccctgaagctcgtgggcgggggccacctgcacgtcaacttcaagaccacatacaagtccaagaaacccgtgaagatgcccggcgtccactacgtggaccgcagactggaaagaatcaaggaggccgacaacgagacctacgtcgagcagtacgagcacgctgtggccagatactccaacctgggcggaggcttcacactc

SEQ ID NO: 18 - protein, 231 aa, Artificial sequence
  Antares
MVSKGEELIKENMRSKLYLEGSVNGHQFKCTHEGEGKPYEGKQTNRIKVVEGGPLPFAFDILATHFMYGSKVFIKYPADLPDYFKQSFPEGFTWERVMVFEDGGVLTATQDTSLQDGELIYNVKVRGVNFPANGPVMQKKTLGWEPSTETMYPADGGLEGRCDKALKLVGGGHLHVNFKTTYKSKKPVKMPGVHYVDRRLERIKEADNETYVEQYEHAVARYSNLGGGFTL

SEQ ID NO: 19 - DNA, 501 bp, Artificial sequence
  Nanoluciferase
gaagatttcgttggggactggcgacagacagccggctacaacctggaccaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggatgatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcggctgtgcgaacgcattctggcgtaa

SEQ ID NO: 20 - protein, 166 aa, Artificial sequence
  Nanoluciferase
EDFVGDWRQTAGYNLDQVLEQGGVSSLFQNLGVSVTPIQRIVLSGENGLKIDIHVIIPYEGLSGDQMGQIEKIFKVVYPVDDHHFKVILHYGTLVIDGVTPNMIDYFGRPYEGIAVFDGKKITVTGTLWNGNKIIDERLINPDGSLLFRVTINGVTGWRLCERILA

SEQ ID NO: 21 - DNA, 516 bp, Artificial sequence
  teLuc
atggtcttcacactcgaagatttcgttggggactggcgacagacagccggctacaacttgagtcaagtccttgaacagggaggtgtgtccagtttgtttcagaatctcggggtgtccgtaactccgatccaaaggattgtcctgagcggtgaaaatgggctgaagatcgacatccatgtcatcatcccgtatgaaggtctgagcggcgaccaaatgggccagatcgaaaaaatttttaaggtggtgtaccctgtggataatcatcactttaaggtgatcctgcactatggcacactggtaatcgacggggttacgccgaacatgatcgactatttcggacggccgtatgaaggcatcgccgtgttcgacggcaaaaagatcactgtaacagggaccctgtggaacggcaacaaaattatcgacgagcgcctgatcaaccccgacggctccctgctgttccgagtaaccatcaacggagtgaccggctggcgtctgcatgaacgcattctggcgtaa

SEQ ID NO: 22 - DNA, 516 bp, Artificial sequence
  yeLuc
atggtcctcactctcgaagatttcgttggggactggcgacagacagacggctataaccaggctcaagtccttgaacagggaggtttgaccagtttgtttcagaacctcggggtgtccgtaactccgatccaaaggattgtcctgagcggtgaaaatgggctgaagatcgacatccatgtcatcatcccgtatgaaggtctgagcggcgaccgaatgggccagatcgaaaaaatttttaaggtggtgtaccctgtggatgatcatcattttaaggtgatcctgcactatggcacactggtaatcgacggggttacgccgaacatgatcgactatttcggacagccgtatgaaggcatcgccgtgttcgacggcaaaaagatcactgtaacagggaccctgtggaacggcaacaaaattatcgacgagcgccggatcaaccccgacggctccctgctgttccgagtaaccatcaacggagtgaccggctggcgtctgagtgaacgcattctggcgtaa

(19) United States
(12) Patent Application Publication
(10) Pub. No.: US 2023/0392182 A1
(43) Pub. Date: Dec. 7, 2023

(54) REPORTER MYCOBACTERIOPHAGE, ASSAYS AND METHODS COMPRISING THE REPORTER MYCOBACTERIOPHAGE

(71) Applicant: Albert Einstein College of Medicine, Bronx, NY (US)

(72) Inventors: William R. Jacobs, Bronx, NY (US); Saranathan Rajagopalan, Bronx, NY (US); Emmanuel Asare, Bronx, NY (US)

(21) Appl. No.: 18/250,685

(22) PCT Filed: Nov. 1, 2021
(86) PCT No.: PCT/US2021/057548

§ 371 Date: Apr. 26, 2023

Related U.S. Application Data
(60) Provisional application No. 63/107,744, filed on Oct. 30, 2020.

(51) Int. Cl. C12Q 1/04; C12N 15/62; C12N 15/86; C12N 9/02; C12Q 1/18

(52) U.S. Cl. CPC C12Q 1/04; C12N 15/62; C12N 15/86; C12N 9/0069; C12Q 1/18; C12R 2001/32

(57) ABSTRACT

A reporter mycobacteriophage comprising a heterologous nucleic acid comprising a promoter-reporter construct encoding a promotor operably linked to a nucleotide sequence encoding a fusion protein, wherein the fusion protein comprises a luciferase protein linked to a fluorescent protein. Methods of utilizing the reporter mycobacteriophage in detection of a viable target microbe in a sample, screening a test substance for treatment of tuberculosis, and detecting a drug-resistant Mycobacterium in a subject are provided.

### FEDERAL RESEARCH STATEMENT

This invention was made with government support under grant number AI026170 awarded by the National Institutes of Health. The government has certain rights in the invention.

### BACKGROUND

A Mycobacterium infection remains as one of the most challenging human health problems in the world, with Mycobacterium tuberculosis (M. tuberculosis) being the most prevalent infectious agent. M. tuberculosis grows very slowly (doubling time of 18 to 24 hours) and the culture-based method of detecting M. tuberculosis infection in an individual is very time-consuming, taking about 4 to 8 weeks for a confirmatory result. Due to its slow growth, it remains challenging to diagnose and perform drug susceptibility testing (DST). Drug resistance in M. tuberculosis is worrisome as resistance towards commercially available antibiotics continues to increase.

Various commercially available DNA-based diagnostic methods have been developed, such as line probe assay (LPA) and GeneXpert. However, these diagnostic methods have limitations, such as, panels for only a limited number of drugs and the need for labor intensive equipment and expensive reagents. In addition, only well characterized resistance mutations are included in the molecular diagnostic panels. Even with the advancements in molecular-based methods, the culture-based method remains the gold standard in the field even though it takes 4 to 8 weeks to obtain a test result.

There thus remains a need for an assay capable of detecting M. tuberculosis which can be conducted in a relatively short period of time, and which can be used to effectively determine drug sensitivity of a M. tuberculosis strain.

### SUMMARY

This disclosure provides a reporter mycobacteriophage comprising a heterologous nucleic acid comprising a promoter-reporter construct encoding a promotor operably linked to a nucleotide sequence encoding a fusion protein, wherein the fusion protein comprises a luciferase protein linked to a fluorescent protein. Also provided is a composition comprising the reporter mycobacteriophage.

This disclosure also provides an assay for detecting a viable target mycobacterial cell in a sample, the assay method comprising: contacting the sample with a reporter mycobacteriophage capable of infecting the mycobacterial cell, wherein the reporter mycobacteriophage comprises a heterologous nucleic acid sequence comprising a promoter-reporter construct encoding a promotor operably linked to a nucleotide sequence encoding a fusion protein, wherein the fusion protein comprises a luciferase protein linked to a fluorescent protein, wherein the fusion protein comprises a luciferase protein and a fluorescent protein; and detecting expression of the fusion protein in the sample, wherein expression of the fusion protein indicates that the viable mycobacterial cell is present in the sample.

This disclosure provides a kit for detection of a mycobacterial cell in a sample, comprising: a reporter mycobacteriophage capable of infecting the target microbe; a substrate for detecting the fusion protein; and instructions for performing the method of detecting a viable mycobacterial cell in a sample as disclosed herein, wherein the reporter mycobacteriophage comprises a heterologous nucleic acid comprising a promoter-reporter construct encoding a promotor operably linked to a nucleotide sequence encoding a fusion protein, wherein the fusion protein comprises a luciferase protein linked to a fluorescent protein.

Provided herein is a method for screening a test substance in vitro for antimycobacterial activity, the method comprising: contacting mycobacterial cells with the test substance to provide treated mycobacterial cells; contacting the treated mycobacterial cells with a reporter mycobacteriophage capable of infecting the mycobacterial cells, wherein the reporter mycobacteriophage comprises a heterologous nucleic acid comprising a promoter-reporter construct encoding a promotor operably linked to a nucleotide sequence encoding a fusion protein, wherein the fusion protein comprises a luciferase protein linked to a fluorescent protein; detecting presence or absence of the fusion protein in the treated mycobacterial cells contacted with the reporter mycobacteriophage, wherein expression of the fusion protein indicates the presence of a viable mycobacterial cell; and determining a percentage inhibition of the treated mycobacterial cells contacted with the reporter mycobacteriophage.

Provided also in the present disclosure is a method for detecting presence of a drug-resistant mycobacterial cell in a sample, the method comprising: providing a sample comprising the mycobacterial cell; contacting the sample with the drug to treat the mycobacterial cell in the sample; adding a reporter mycobacteriophage capable of infecting the mycobacterial cell to the sample, wherein the reporter mycobacteriophage comprises a heterologous nucleic acid comprising a promoter-reporter construct encoding a promotor operably linked to a nucleotide sequence encoding a fusion protein, wherein the fusion protein comprises a luciferase protein linked to a fluorescent protein; and detecting expression of the fusion protein in the sample, wherein expression of the fusion protein indicates that the treated mycobacterial cell is resistant to the drug.

The above described and other features are exemplified by the following figures and detailed description.

### BRIEF DESCRIPTION OF THE DRAWINGS

FIG. 1A is a schematic representation of phAE1142 shuttle phasmid carrying the mNeon_green-nLuc cassette.

FIG. 1B is a schematic representation of phAE1158 shuttle phasmid carrying the Antares2_nLuc cassette.

FIGS. 2A to 2I show the results of drug response curve assay using phAE1142. The drugs isoniazid (INH) and rifampicin (RIF) were tested against the M. tuberculosis strains mc²6230 (FIGS. 2A-2C), mc²8245 (FIGS. 2D-2F), and mc²8247 (FIGS. 2G-2I). The M. tuberculosis cells were added to wells (10⁵/well) and incubated to 48 hours with varying concentrations of each drug followed by 24 hour infection with 10⁵ plaque forming units (PFU) of phAE1142 phage. Furimazine substrate was added to the wells and the image was captured using a Bronx box 3.0, with a 2 minute exposure. Plate images are shown in FIG. 2A (strain mc²6230), FIG. 2D (strain mc²8245), and FIG. 2G (strain mc²8247). Dose response curves are shown in FIGS. 2B-2C (strain mc²6230), in FIGS. 2E-2F (strain mc²8245), and FIGS. 2H-2I (strain mc²8247).

FIGS. 3A and B are graphs showing the limit of detection of (LOD) M. tuberculosis strain mc²6230 by phAE1142 at 24 hours (FIG. 3A) and 48 hours (FIG. 3B) after infection. The results show phAE1142 mediated M. tuberculosis limit of detection is <100 CFU at 24 h, and signal improves with increased incubation time.

### DETAILED DESCRIPTION

Disclosed herein is a reporter mycobacteriophage comprising a highly sensitive reporter cassette that can be used for the rapid detection of mycobacteria in clinical samples, for determining the susceptibility of the mycobacteria to antibiotics. The reporter mycobacteriophage can also be effectively utilized to assess the minimum inhibitory concentration of a test substance (e.g., a drug). The disclosed reporter mycobacteriophage emits at least one thousand times more luminescence as compared to firefly luciferase and at least ten times more luminescence as compared to nanoluciferase, making it a powerful system for the detection of mycobacteria. Further, the reporter mycobacteriophage is highly sensitive, capable of detecting less than one hundred mycobacteria cells in a sample. The reporter mycobacteriophage also allows for rapid high throughput detection of viable mycobacteria in a sample without the need for labor-intensive instruments, expensive reagents, or extensive training.

A “heterologous nucleic acid” or “heterologous gene”, with regard to its presence in a mycobacteriophage or vector backbone, refers to nucleic acid that is not naturally present in the mycobacteriophage or vector backbone, respectively, or, if from the same source, is modified from its original form. The heterologous nucleic acid can be a DNA sequence including a heterologous gene.

A “nucleic acid”, “nucleic acid sequence”, or “nucleotide sequence” refers to a polymeric form of nucleotides.

A “mycobacteriophage” is a phage capable of infecting one or more Mycobacterial strains. A mycobacteriophage containing heterologous nucleic acid in its genome is also be referred to herein interchangeably as a “recombinant bacteriophage” or a “recombinant mycobacteriophage,” respectively. An “isolated” recombinant mycobacteriophage is one that is not naturally occurring and is separate from the bacterial cell in which it replicates.

A DNA segment, or nucleic acid, is “operably linked” when placed into a functional relationship with another DNA segment. For example, DNA for a promoter is operably linked to a coding sequence if it stimulates the transcription of the sequence. In general, DNA sequences that are operably linked are contiguous, and can be both contiguous and in reading phase. Linking is accomplished by ligation at convenient restriction sites or at adapters or linkers inserted in lieu thereof.

The term “promoter” refers to a minimal nucleotide sequence which is sufficient to direct transcription of a particular gene. The promoter includes the core promoter, which is the minimal portion of the promoter required to properly initiate transcription and can also include regulatory elements such as transcription factor binding sites. In the present disclosure, the promoter controls expression of a fusion protein as a detectable marker (reporter protein).

A “promoter-reporter construct” or “reporter cassette” refers to a nucleic acid sequence encoding a promoter operably linked to a reporter gene (e.g., a gene encoding a reporter protein). The promoter drives the transcription of the reporter gene, which in turn, is translated into a reporter protein. The “reporter” or “reporter protein” is a protein whose expression is correlated with a cellular event such as infection, etc. The expression of a reporter protein can be measured using various methods and depends on the type of reporter protein that is expressed. In the present disclosure, the reporter is a fusion protein.

A “fusion protein” is a protein encoded by two or more separate genes which have been joined together so that they are transcribed and translated as a single unit to produce a single polypeptide. A fusion protein generally has all or a substantial portion of a first polypeptide, linked at the N- or C-terminus, to all or a portion of a second polypeptide. The fusion proteins described herein are comprise, consist essentially of, or consist of at least two proteins (at least one luciferase protein and at least one fluorescent protein) fused end to end with an optional amino acid linker there between.

An “amino acid variant” refers to a protein having a change in the amino acid sequence of the encoded protein relative to the original amino acid sequence. The amino acid variation is the result of a base change in the nucleic acid sequence of the encoded protein that leads to a change in the amino acid sequence of the protein. In the present disclosure, an amino acid variant of a luciferase protein or a fluorescent protein increases or has a neutral effect on the activity of the protein, i.e., there is no negative impact on the protein's activity.

The present disclosure provides a reporter mycobacteriophage comprising a heterologous nucleic acid encoding a promotor operably linked to a nucleic acid sequence encoding a fusion protein, wherein the fusion protein comprises a luciferase protein linked to a fluorescent protein. In an aspect, the fusion protein is a reporter protein.

The reporter mycobacteriophage comprises a vector backbone into which the heterologous nucleic acid encoding a promoter and the fusion protein are integrated. As used herein, a “vector backbone” refers to a nucleic acid molecule capable of transporting one or more other nucleic acid to which it has been linked. In the present disclosure, the vector backbone is derived from a mycobacteriophage. The vector backbone comprises one or more nucleic acid sequences that are naturally present in the mycobacteriophage genome sequence, but the complete sequence is not identical to the naturally occurring mycobacteriophage genome sequence. The vector backbone can be made by one or more of a mutation, insertion and/or deletion of the mycobacteriophage genome sequence, or by one or more subsequent mutation(s), insertion(s) and/or deletion(s) of a mutated, deleted and or inserted mycobacteriophage genome sequence. Accordingly, a vector backbone derived from a mycobacteriophage as used herein includes those both directly derived from the naturally occurring mycobacteriophage and those which are subsequently derived. In an aspect, the vector backbone comprises a mycobacteriophage genomic sequence having a deletion in a non-essential area thereof (i.e., not essential for replication) permitting insertion of the heterologous nucleic acid.

In particular, the vector backbone is a mycobacteriophage which is capable of infecting and/or lysing at least one strain of Mycobacterium. Different mycobacteriophages have varying host specificities. In an aspect, the mycobacteriophage selected as the vector backbone is specific for a single Mycobacterium strain or a plurality of Mycobacterium strains. In an aspect, the mycobacteriophage selected as the backbone for the reporter mycobacteriophage is capable of infecting and/or lysing a plurality of Mycobacterium strains. Examples of the Mycobacterium strain include Mycobacterium. tuberculosis, Mycobacterium bovis, Mycobacterium smegmatis, Mycobacterium bovis-BCG, Mycobacterium avium, Mycobacterium phlei, Mycobacterium fortuitum, Mycobacterium lufu, Mycobacterium paratuberculosis, Mycobacterium habana, Mycobacterium scrofulaceum, Mycobacterium intracellularae, or a combination thereof. In an aspect, the mycobacteriophage is capable of infecting and/or lysing only M. tuberculosis. Examples of known mycobacteriophages include DS6A, L5, TM4, and D29. However, the mycobacteriophage used as the vector backbone is not limited to these examples, and any mycobacteriophage capable of infecting a target Mycobacterium can be used.

The mycobacteriophage can be a conditionally replicating mycobacteriophage. For example, the mycobacteriophage can be a temperature sensitive mutant, which is capable of maximum replication in Mycobacterium at a first temperature (permissive temperature) and replicates to a substantially lesser degree, or not at all, at a second temperature (non-permissive temperature). The second non-permissive temperature can be either higher or lower than the first temperature. In an aspect, the permissive temperature is 21° C. to 30′C, and the non-permissive temperatures is 37° C. to 42° C. For example, thermosensitive mutations can be present in the mycobacteriophage that allow replication/propagation in Mycobacterium at temperatures of 30° C., but not at higher temperatures of 37° C. (e.g., mycobacteriophage TM4) or 38.5° C. (e.g., mycobacteriophage D29).

The insertion site of the heterologous nucleic acid can occur within any non-essential region of the mycobacteriophage vector backbone. The heterologous nucleic acid includes a promoter-reporter construct comprising a promotor sequence upstream of and operably linked to a nucleic acid sequence encoding a fusion protein. In an aspect, the promoter comprises the promoter from a mycobacteriophage such as DS6A, L5, TM4, D29, or a combination thereof. In an aspect, the promoter is from the L5 mycobacteriophage, and has the nucleic acid sequence of SEQ ID NO:1.

In addition to the promoter-reporter construct, other useful elements can also be included in the heterologous nucleic acid. For example, the heterologous nucleic acid may further comprise an antibiotic resistance gene, a bacterial origin of replication (e.g., OriE), a mycobacteriophage origin of replication (e.g., OriM), and/or a lambda phage cos sequence. The heterologous nucleic acid can also include other sequences such as an Escherichia coli cosmid sequence, a mycobacteriophage integration sequence, a targeting or localization sequence, a tag sequence, and/or a sequence of one or more other fluorescent proteins that are not part of the fusion protein. A combination comprising one or more of these elements can also be present.

The antibiotic resistance gene is not limited, and includes for example, the genes encoding resistance to ampicillin, kanamycin, spectinomycin, streptomycin, apramycin, hygromycin, carbenicillin, bleomycin, erythromycin, polymyxin B, tetracycline, and/or chloramphenicol. In an aspect, the antibiotic resistance gene comprises an ampicillin resistance gene.

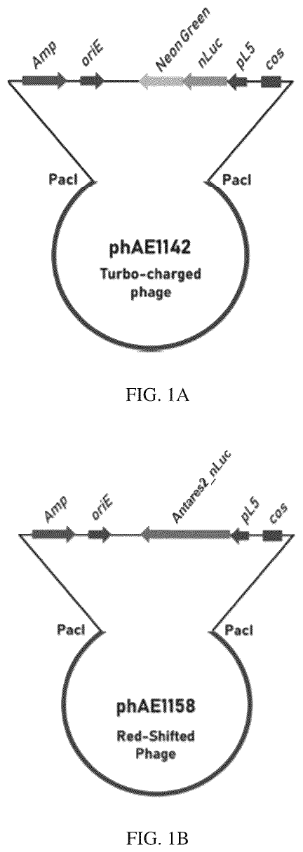

In an aspect, the heterologous nucleic acid sequence comprises a lambda phage cos sequence, an L5 promoter sequence operably linked to a nucleic acid sequence encoding the fusion protein, an OriE sequence and/or an OriM sequence, and an antibiotic resistance gene sequence. Exemplary reporter mycobacteriophages phAE1142 and phAE1158 including the heterologous nucleic acid sequence, are shown in FIGS. 1A and 1B, respectively.

The nucleic acid sequence encoding the fusion protein comprises the nucleic acid sequence of a luciferase protein linked to the nucleic acid sequence of a fluorescent protein. The nucleic acid sequence of the fluorescent protein can be inserted upstream and/or downstream of the nucleic acid sequence for the luciferase protein. The fluorescent protein can thus be attached to the N-terminus, the C-terminus, or both the N-terminus and the C-terminus of the luciferase protein. When the fusion protein is expressed, the luciferase protein and the fluorescent protein can be connected directly to each other by a peptide bond or can be separated by a short linker amino acid sequence (e.g., less than 10 amino acids). The linker amino acid sequence is encoded by a nucleic acid inserted between the gene encoding the luciferase protein and the gene encoding the fluorescent protein.

The fusion protein can include sequences from multiple fluorescent proteins, multiple luciferase proteins, amino acid variants thereof, other selected proteins, or a combination thereof. In an aspect, the fusion protein includes multiple copies (e.g., more than one) of the fluorescent protein attached to the N-terminus, the C-terminus, or both the N-terminus and the C-terminus of the luciferase protein. In an aspect, the fusion protein comprises a copy of the fluorescent protein attached to the N-terminus of the luciferase protein and a copy of the fluorescent protein attached to the C-terminus of the fluorescent protein. In an aspect, a first fluorescent protein is connected to the N-terminus of the luciferase protein and a second fluorescent protein is connected to the C-terminus of the luciferase protein. In an aspect, the fusion protein comprises a first fluorescent protein upstream of the luciferase protein and a second fluorescent protein downstream of the luciferase protein. The first fluorescent protein can be the same as or different from the second fluorescent protein connected to the C-terminus of the luciferase protein.

A “luciferase protein” refers to an enzyme which produces bioluminescence upon oxidation of a suitable substrate (a luciferin). The luciferase protein can comprise those found in Gaussia, Coleoptera, (e.g., fireflies), Renilla, Vargula, Oplophorus, mutants thereof, portions thereof, variants thereof, and any other luciferase enzymes suitable for the systems and methods described herein. In an aspect, the luciferase protein is the nanoluciferase protein. As used herein “nanoluciferase” or “NanoLuc” or “nLuc” are used interchangeably to refer to the luciferase enzyme from Oplophorus gracilirostris (e.g., NANOLUC™; from Promega Corporation), a 19.1 kDa, ATP-independent luciferase that utilizes the coelenterazine analog furimazine to produce high intensity glow, and having the nucleic acid sequence of SEQ ID NO:19 and the amino acid sequence of SEQ ID NO:20.

In an aspect, the luciferase protein comprises a nanoluciferase, an amino acid sequence variant thereof, or a combination thereof. In an aspect, an amino acid sequence variant of nanoluciferase comprises teLuc, yeLuc, LumiLuc, or a combination thereof. However, the amino acid sequence variant of the nanoluciferase protein is not limited thereto and any amino acid variant which retains the ability to act on a substrate can be used. The gene sequences for teLuc and yeLuc are deposited to GenBank under the accession numbers KX963378 and KX963379, respectively. The nucleic acid sequence of teLuc is provided herein as SEQ ID NO:21 and the nucleic acid sequence of yeLuc is provided herein as SEQ ID NO:22. LumiLuc is an enzyme with broad substrate specificity derived from teLuc, and is described in Hsien-Wei Yeh, et al, ACS Chem. Biol., 2019, 14(5): 959-965.

In an aspect, the luciferase protein comprises nanoluc (nLuc), teLuc, yeLuc, LumiLuc, an amino acid variant thereof, or a combination thereof.

A “fluorescent protein” refers to a protein capable of emitting light when excited with appropriate electromagnetic radiation. The fluorescent protein can be a naturally occurring protein or an amino acid variant of a naturally occurring protein. In an aspect, the fluorescent protein comprises monomeric green fluorescent protein (mNeonGreen), moxNeonGreen (an oxidizing variant of mNeonGreen), mTourquoise, mTourquoise2, cyan-excitable orange fluorescent protein (CyOFP), cyan-excitable red fluorescent protein (CyRFP), monomeric cyan-excitable red fluorescent protein (mCyRFP1), tdTomato, mCherry, mApple, mCardinal, mMaroon, mScarlett, mWassabi, an amino acid variant thereof, or a combination thereof.

In an aspect, the heterologous nucleic acid comprises a promoter-reporter construct comprising an L5 promoter operably linked to the nucleic acid sequence encoding the fusion protein. In an aspect, the fusion protein comprises mNeonGreen and nLuc, mTurquoise and nLuc, CyOFP and nLuc, CyOFP and teLuc, or CyOFP and yeLuc. One or multiple copies of each protein can be present in the fusion protein.

In an aspect, the fusion protein comprises mNeonGreen and nLuc (mNeonGreen-nLuc). In an aspect, the fusion protein comprises mNeonGreen-nLuc, and has a nucleic acid sequence identical to (100% identity) SEQ ID NO: 3 and/or an amino acid sequence identical to SEQ ID NO:4. In an aspect, the fusion protein has a nucleic acid sequence which is at least 90% identical to SEQ ID NO:3, at least 95% identical to SEQ ID NO:3, or at least 99% identical to SEQ ID NO:3. In an aspect, the fusion protein has an amino acid sequence which is at least 90% identical to SEQ ID NO:4, at least 95% identical to SEQ ID NO:4, or at least 99% identical to SEQ ID NO:4.

In an aspect, the fusion protein comprises mTurquoise and nLuc (mTurquoise-nLuc). In an aspect, the fusion protein comprises mTurquoise-nLuc and the fusion protein has a nucleic acid sequence identical to SEQ ID NO: 5 and/or an amino acid sequence identical to SEQ ID NO:6. In an aspect, the fusion protein has a nucleic acid sequence which is at least 90% identical to SEQ ID NO:5, at least 95% identical to SEQ ID NO:5, or at least 99% identical to SEQ ID NO:5. In an aspect, the fusion protein has an amino acid sequence at least 9% identical to SEQ ID NO:6, at least 95% identical SEQ ID NO:6, or at least 99% SEQ ID NO:6.

In an aspect, the fusion protein comprises nLuc sandwiched between a first copy of mNeonGreen and a second copy of mNeonGreen (mNeonGreen-nLuc-mNeonGreen). In an aspect, the fusion protein comprises mNeonGreen-nLuc-mNeonGreen, and has a nucleic acid sequence identical to SEQ ID NO:7 and/or an amino acid sequence identical to SEQ ID NO:8. In an aspect, the fusion protein has a nucleic acid sequence which is at least 90% identical to SEQ ID NO:7, at least 95% identical to SEQ ID NO:7, or at least 99% identical to SEQ ID NO:7. In an aspect, the fusion protein has an amino acid sequence which is at least 90% identical to SEQ ID NO:8, at least 95% identical to SEQ ID NO:8, or at least 99% identical to SEQ ID NO:8.

In an aspect, the fusion protein comprises nLuc sandwiched between mNeonGreen upstream and mTurquoise downstream of the nLuc (mNeonGreen-nLuc-mTurquoise). In an aspect, the fusion protein comprises mNeonGreen-nLuc-mTurquoise and has a nucleic acid sequence identical to SEQ ID NO: 9 and/or an amino acid sequence identical to SEQ ID NO:10. In an aspect, the fusion protein has a nucleic acid sequence which is at least 90% identical to SEQ ID NO:9, at least 95% identical to SEQ ID NO:9, or at least 99% identical to SEQ ID NO:9. In an aspect, the fusion protein has an amino acid sequence which is at least 90% identical to SEQ ID NO:10, at least 95% identical to SEQ ID NO:10, or at least 99% identical to SEQ ID NO:10.

In an aspect, the fusion protein comprises nLuc sandwiched between mTurquoise upstream and mNeonGreen downstream of the nLuc (mTurquoise-nLuc-mNeon_green). In an aspect, the fusion protein comprises mTurquoise-nLuc-mNeon_green, and has a nucleic acid sequence identical to SEQ ID NO: 11 and/or an amino acid sequence identical to SEQ ID NO:12. In an aspect, the fusion protein has a nucleic acid sequence which is at least 90% identical to SEQ ID NO:11, at least 95% identical to SEQ ID NO:11, or at least 99% identical to SEQ ID NO:11. In an aspect, the fusion protein has an amino acid sequence which is at least 90% identical to SEQ ID NO:12, at least 95% identical to SEQ ID NO:12, or at least 99% identical to SEQ ID NO:12.

In an aspect, the fusion protein comprises nLuc between a first copy of mTurquoise and a second copy of mTurquoise (mTurquoise-nLuc-mTurquoise). In an aspect, the fusion protein comprises mTurquoise-nLuc-mTurquoise, and has a nucleic acid sequence identical to SEQ ID NO: 13 and/or an amino acid sequence identical to SEQ ID NO:14. In an aspect, the fusion protein has a nucleic acid sequence which is at least 90% identical to SEQ ID NO:13, at least 95% identical to SEQ ID NO:13, or at least 99% identical to SEQ ID NO:13. In an aspect, the fusion protein has an amino acid sequence which is at least 90% identical to SEQ ID NO:14, at least 95% identical to SEQ ID NO:14, or at least 99% identical to SEQ ID NO:14.

In an aspect, the fusion protein comprises teLuc between a first copy of CyOFP and a second copy of CyOFP (CyOFP-teLuc-CyOFP; also known as “Antares2”). In an aspect, the fusion protein comprises CyOFP-teLuc-CyOFP, and has a nucleic acid sequence identical to SEQ ID NO: 15 and/or an amino acid sequence identical to SEQ ID NO:16. In an aspect, the fusion protein has a nucleic acid sequence which is at least 90% identical to SEQ ID NO:15, at least 95% identical to SEQ ID NO:15, or at least 99% identical to SEQ ID NO:15. In an aspect, the fusion protein has an amino acid sequence which is at least 90% identical to SEQ ID NO:16, at least 95% identical to SEQ ID NO:16, or at least 99% identical to SEQ ID NO:16.

In an aspect, the fusion protein comprises NanoLuc between a first copy of CyOFP and a second copy of CyOFP (CyOFP-nLuc-CyOFP; also known as “Antares”). In an aspect, the fusion protein comprises CyOFP-nLuc-CyOFP, and has a nucleic acid sequence identical to SEQ ID NO: 17 and/or an amino acid sequence identical to SEQ ID NO:18. In an aspect, the fusion protein has a nucleic acid sequence which is at least 90% identical to SEQ ID NO:17, at least 95% identical to SEQ ID NO:17, or at least 99% identical to SEQ ID NO:17. In an aspect, the fusion protein has an amino acid sequence which is at least 90% identical to SEQ ID NO:18, at least 95% identical to SEQ ID NO:18, or at least 99% identical to SEQ ID NO:18.

In an aspect, the fusion protein has a nucleic acid sequence which is at least 90% identical to SEQ ID NO:3, SEQ ID NO:5, SEQ ID NO:7, SEQ ID NO:9, SEQ ID NO:11, SEQ ID NO:13, SEQ ID NO:15, or SEQ ID NO:17. In an aspect, the fusion protein has an amino acid sequence which is at least 90% identical to SEQ ID NO:4, SEQ ID NO:6, SEQ ID NO:8, SEQ ID NO:10, SEQ ID NO:12, SEQ ID NO:14, SEQ ID NO:16, or SEQ ID NO:18.

In an aspect, the fusion protein has a nucleic acid sequence which is at least 95% identical to SEQ ID NO:3, SEQ ID NO:5, SEQ ID NO:7, SEQ ID NO:9, SEQ ID NO:11, SEQ ID NO:13, SEQ ID NO:15, or SEQ ID NO:17. In an aspect, the fusion protein has an amino acid sequence which is at least 95% identical to SEQ ID NO:4, SEQ ID NO:6, SEQ ID NO:8, SEQ ID NO:10, SEQ ID NO:12, SEQ ID NO:14, SEQ ID NO:16, or SEQ ID NO:18.

In an aspect, the fusion protein has a nucleic acid sequence which is at least 99% identical to SEQ ID NO:3, SEQ ID NO:5, SEQ ID NO:7, SEQ ID NO:9, SEQ ID NO:11, SEQ ID NO:13, SEQ ID NO:15, or SEQ ID NO:17. In an aspect, the fusion protein has an amino acid sequence which is at least 99% identical to SEQ ID NO:4, SEQ ID NO:6, SEQ ID NO:8, SEQ ID NO:10, SEQ ID NO:12, SEQ ID NO:14, SEQ ID NO:16, or SEQ ID NO:18.

In an aspect, the fusion protein has a nucleic acid sequence which is identical to SEQ ID NO:3, SEQ ID NO:5, SEQ ID NO:7, SEQ ID NO:9, SEQ ID NO:11, SEQ ID NO:13, SEQ ID NO:15, or SEQ ID NO:17. In an aspect, the fusion protein has an amino acid sequence which is identical to SEQ ID NO:4, SEQ ID NO:6, SEQ ID NO:8, SEQ ID NO:10, SEQ ID NO:12, SEQ ID NO:14, SEQ ID NO:16, or SEQ ID NO:18.

The reporter mycobacteriophage encoding the fusion protein can be utilized as a reporter system based on Bioluminescence Resonance Energy Transfer (BRET). The luciferase protein is selected to function together with the fluorescent protein as a BRET pair. In the presence of an appropriate substrate for the luciferase protein, the luciferase-catalyzed biochemical reaction emits light or “bioluminescence”. The fluorescent protein acts as an acceptor molecule to accept the bioluminescence generated by the luciferase protein. When the emission spectrum of the bioluminescence from the luciferase protein partially overlaps the absorption spectrum of the fluorescent protein, the bioluminescence emitted by the luciferase-catalyzed biochemical reaction excites the fluorescent protein, which in turn, emits photons. The luciferase protein thus acts as an energy transfer donor and the fluorescent protein acts as an energy transfer acceptor.

Upon infection of a mycobacterial cell with the reporter mycobacteriophage, activation of the promoter induces transcription of the nucleic acid encoding the fusion protein, which is in turn translated into protein thereby resulting in expression of the fusion protein. The reporter mycobacteriophage infects and induces the expression of the fusion protein in a viable mycobacterial cell, whereas a non-viable mycobacterial cell does not support replication of the reporter mycobacteriophage. Viability of a mycobacterial cell can thus be determined by detecting the expression of the fusion protein in the mycobacterial cell and/or in a sample containing the mycobacterial cell.

To facilitate infection of a mycobacterial cell, the reporter mycobacteriophage is placed in contact with the mycobacterial cell under conditions which facilitate infection of the cell with the reporter mycobacteriophage. In an aspect, the reporter mycobacteriophage is placed in contact with the mycobacterial cell at a temperature and for a time period to facilitate infection of a viable mycobacterial cell. For example, the contacting can occur at temperature of about 30° C. to about 37° C. or about 32° C. to about 37° C., for a time period of about 4 hours to about 36 hours, or about 4 hours to about 24 hours, or about 4 hours to about 18 hours, or about 4 hours to about 12 hours, or about 8 hours to about 24 hours, or about 8 hours to about 18 hours, or about 8 hours to about 12 hours. In an aspect, the sample is incubated with the reporter mycobacteriophage at a for a time period of about 4 hours, about 8 hours, about 12 hours, about 18 hours, about 24 hours, or about 36 hours. In an aspect, the sample is incubated with the reporter mycobacteriophage at a temperature of about 37° C., for a time period of about 4 hours to about 12 hours.

Once the mycobacterial cell has been infected with the reporter mycobacteriophage, expression of the fusion protein can be measured by the addition of a substrate for the luciferase protein. Contact between the substrate and the fusion protein results in oxidation of the substrate by the luciferase and emission of bioluminescence, the fluorescent protein then acts as an acceptor molecule to accept the bioluminescence generated by the luciferase protein, and subsequently emits fluorescent light. The amount of fluorescent light emitted is used as a direct measure of the expression of the fusion protein. Since the emission of fluorescent light only occurs when the reporter mycobacteriophage infects a mycobacterial cell, the emission of fluorescent light can be attributed to the presence of a viable mycobacterial cell in a given sample at the time of infection. The amount of fluorescent light emitted from a sample can also be correlated with the number of viable mycobacterial cells originally present in the sample.

The emission of fluorescent light can be measured at a single point in time or over an extended period of time, following the addition of the substrate. The time period between the addition of the substrate for the luciferase protein and measurement of the emitted light (fluorescent and/or luminescent light) can be adjusted as needed to ensure that maximum light emission is measured.

In an aspect, detection of the fusion protein comprises contacting a substrate with the infected mycobacterial cell, reacting the substrate with the fusion protein present in the infected mycobacterial cell, and measuring an amount of fluorescent light produced/emitted by the infected mycobacterial cell. In an aspect, the substrate comprises coelenterazine, furimazine, hydrofurimazine, fluorofurimazine, selenoterazine, diphenylterazine, a derivative thereof, or a combination thereof. However, the substrate is not necessarily limited to these compounds. The time period between the addition of the substrate for the luciferase protein and measurement of the emitted light (fluorescent and/or luminescent light) can be determined by those of skill in the art without undue experimentation. In an aspect, the time period between the addition of the substrate for the luciferase protein and measurement of the emitted light can be about 30 seconds to about 60 minutes, or about 1 minute to about 30 minutes, or about 5 minutes to about 20 minutes, or about 10 minutes to about 20 minutes, or about 12 minutes to about 18 minutes.

Measuring the amount of the light emitted by the infected mycobacterial cell can comprise any method suitable for detecting and quantifying fluorescent light. In an aspect, light emitted by a sample comprising the infected mycobacterial cell can be measured. Expression of the fusion protein can be detected using a luminometer or a photographic imaging box.

In an aspect, expression of the fusion protein in a sample comprising an infected mycobacterial cell is measured using a photographic imaging box. The photographic imaging box is temperature controlled photography box which captures the generation of fluorescent light in real time. In an aspect, the photographic imaging box is the Bronx Box 3 (Riska et al, J. Clin. Microbiol., (1991) vol. 37, no. 4, pp. 1144-1149). The Bronx Box 3 is a custom temperature controlled photography box which takes photographs of luminescent cell growth. The frame of the box is constructed out of slotted black aluminum rails and the walls are made of black hardboard panels. Inside the box there is a digital single-lens reflex (DSLR) camera mounted overhead, a heater, a fan, and a temperature sensor. The heater is capable of heating the box from ambient conditions to about 37° C.±1° C. in 20 minutes and has a maximum operating temperature of about 48° C. One or more fans mounted within the box ensure a consistent temperature throughout the interior. A proportional-integral-derivative (PID) controller and a 100 ohm (Ω) Resistance Temperature Detector (RTD) are used to set and monitor the temperature within the box. The camera is equipped with a 100 mm f/2.8 macro lens and faces towards the base of the copy stand where a single or multiwell plate (e.g., a 96 well plate) can be incubated and imaged concurrently. The camera and temperature can be controlled from outside the box to minimize disruptions to the sample and the interior temperature while the plate is incubating. All electronics, including the PID and fuses, are housed in an enclosure outside of the box.

Alternatively, or in addition to, expression of the fusion protein in infected mycobacterial cells can be measured by, for example, fluorescence activated cell sorting (FACS) or fluorescent microscopy. The detection methods disclosed herein are not limited thereto and any method suitable for measuring fluorescent light in a sample or in a viable target microbe can be used.

In an aspect, the present disclosure provides a composition comprising the reporter mycobacteriophage. The composition can comprise materials such as a stabilizer, a mycobacterial growth medium, a buffer, cesium chloride, a high salt buffer, trehalose, skimmed milk, or a combination thereof.

The advantageous aspects conferred by the reporter mycobacteriophages of the present disclosure can be utilized to detect for the presence of a viable target microbe (e.g. a Mycobacterium) in a sample, to determine efficacy of a test substance for treatment of tuberculosis, and to determine whether a subject is infected with a drug-resistant Mycobacterium strain.

The present disclosure provides a method for detecting a viable mycobacterial cell in a sample, the method comprising:
contacting the sample with a reporter mycobacteriophage capable of infecting the mycobacterial cell, wherein the reporter mycobacteriophage comprises a heterologous nucleic acid comprising a promoter-reporter construct encoding a promotor operably linked to a nucleotide sequence encoding a fusion protein, wherein the fusion protein comprises a luciferase protein linked to a fluorescent protein, wherein the fusion protein comprises a luciferase protein and a fluorescent protein; and
detecting expression of the fusion protein, wherein expression of the fusion protein indicates that the viable mycobacterial cell is present in the sample.

In an aspect, the target microbe comprises a Mycobacterium. In an aspect, the Mycobacterium comprises M. tuberculosis, M. bovis, M. smegmatis, M. bovis-BCG, M. avium, M. phlei, M. fortuitum, M. lufu, M. paratuberculosis, M. habana, M. scrofulaceum, M. intracellularae, or a combination thereof. In an aspect, the Mycobacterium comprises M. tuberculosis, M. bovis, M. smegmatis, M. bovis-BCG, or a combination thereof. In an aspect, the Mycobacterium comprises M. tuberculosis. However, the target microbe is not limited to those listed above and can be any Mycobacterium that can be infected with the reporter mycobacteriophage.

In an aspect, contacting the sample with the reporter mycobacteriophage comprises infecting a viable target microbe present in the sample with the reporter mycobacteriophage and inducing expression of the fusion protein in the target microbe. Expression of the fusion protein can be measured by measuring fluorescence in the sample and comparing to background levels (autofluorescence) present in a negative control sample. For example, the negative control sample can contain the reporter mycobacteriophage without any viable microbial cells, or the reporter mycobacteriophage in the presence of non-viable (e.g. lysed) microbial cells. A viable mycobacterial cell present in the sample will be infected by the reporter mycobacteriophage and express the fusion protein, whereas a non-viable mycobacterial cell will not support replication of the reporter mycobacteriophage, the fusion protein will not be expressed, and specific fluorescence will not be detected.

The sample may be treated to promote infection of a viable mycobacterial cell by the reporter mycobacteriophage. In an aspect, an amount of the reporter mycobacteriophage is added to the sample and the sample is incubated at a temperature and for a time period to facilitate infection of any viable mycobacterial cell present in the sample. The conditions facilitating infection of a viable mycobacterial cell with the reporter mycobacteriophage comprise those previously described herein. In an aspect, the sample is incubated with the reporter mycobacteriophage at a temperature of about 37° C., for a time period of about 4 hours to about 24 hours.

In an aspect, detecting expression of the fusion protein comprises adding a substrate to the sample, reacting the substrate with fusion protein present in the sample, and measuring an amount of fluorescent light emitted from the sample. The fluorescent light can be detected as previously described.

The sample can be any material suspected of containing or known to contain the target microbe. In an aspect, the sample is a sample suspected of containing a Mycobacterium. The sample can comprise a biological sample, an environmental sample, a lab sample, a stock sample, a manufacturing sample, a vaccine, or a combination thereof. In an aspect, the test sample is a biological sample from a subject. The subject can be a mammalian subject, and specifically, a human or animal subject. In an aspect, the biological sample is from a human subject. The biological sample can comprise sputum, blood, throat swab, genital swab, urethral swab, body fluids (CSF and others) or a combination thereof.

Processing of the sample prior to conducting the assay can be performed to ensure that there are no factors present which may prevent the reporter mycobacteriophage from infecting a viable target microbe. Methods of preliminary processing of biological samples are described, for example, in Jacobs, W. R., Jr., et al. (Rapid assessment of drug susceptibilities of Mycobacterium tuberculosis by means of luciferase reporter phages. Science, 1993. 260(5109): p. 819-22) and U.S. Pat. No. 9,447,449. The type of processing is not limited, as long as it does not alter the viability of the target microbe in the sample and does not interfere with the interaction between the reporter mycobacteriophage and the target microbe.

The present disclosure provides a kit for detection of a target microbe in a sample. In an aspect, the kit comprises a reporter mycobacteriophage which is capable of infecting a target microbe, a substrate for detecting the fusion protein, and instructions for the above described assay for detecting a viable target microbe in the sample. The reporter mycobacteriophage is described herein and comprises a heterologous nucleic acid comprising a promoter-reporter construct encoding a promotor operably linked to a nucleotide sequence encoding a fusion protein, wherein the fusion protein comprises a luciferase protein linked to a fluorescent protein.

Packaging for the kits and written instructions on how to utilize the kit, are also provided. Additional components can also be included in the kit as desired, such as, for example, wash buffers, growth medium, and/or receptacles for conducting the assay (e.g., multi-well plates, test tubes).

The above-described methods, assay, and aspects thereof, can be applied in each of the methods provided in the present disclosure.

The present disclosure provides a method for screening a test substance in vitro for antimycobacterial activity, the method comprising:
contacting mycobacterial cells with the test substance to provide treated mycobacterial cells;
contacting the treated mycobacterial cells with a reporter mycobacteriophage capable of infecting the treated mycobacterial cells, wherein the reporter mycobacteriophage comprises a heterologous nucleic acid comprising a promoter-reporter construct encoding a promotor operably linked to a nucleotide sequence encoding a fusion protein, wherein the fusion protein comprises a luciferase protein linked to a fluorescent protein;
detecting expression of the fusion protein in the treated mycobacterial cells contacted with the reporter mycobacteriophage, wherein expression of the fusion protein indicates that a viable mycobacterial cell is present in the sample; and
determining a percentage inhibition of the treated mycobacterial cells contacted with the reporter mycobacteriophage.

The mycobacterial cells used to screen the test substance can comprise Mycobacterium tuberculosis, Mycobacterium bovis, Mycobacterium smegmatis, Mycobacterium bovis-BCG, Mycobacterium avium, Mycobacterium phlei, Mycobacterium fortuitum, Mycobacterium lufu, Mycobacterium paratuberculosis, Mycobacterium habana, Mycobacterium scrofulaceum, Mycobacterium intracellularae, or a combination thereof. In an aspect, the mycobacterial cells comprise Mycobacterium tuberculosis (M. tuberculosis). In an aspect, the M. tuberculosis is a multidrug resistant tuberculosis (MDR-TB) strain, an extensively drug resistant tuberculosis (XDR-TB) strain, or a combination thereof. In an aspect, the M. tuberculosis strain is resistant to, or is suspected of being resistant to kanamycin, isoniazid, rifampicin, or a combination thereof.

In an embodiment the test substance is an organic small molecule having a size of 2000 Da or less. In an embodiment the test substance is an organic small molecule having a size of 1500 Da or less. In an aspect, the test substance is an antibiotic. The antibiotic can be a known antibiotic, a suspected antibiotic, or a combination thereof. In an aspect, the test substance comprises an aminoglycoside (e.g., amikacin), a polypeptide (e.g., capreomycin, viomycin, enviomycin), a fluoroquinolone, (e.g., ciprofloxacin, levofloxacin, moxifloxacin), a thioamide (e.g. ethionamide), or a combination thereof. The test substance is not limited to these substances, and any other test substance can be tested for in vitro efficacy in the same manner.

The contacting of the mycobacterial cells with the test substance comprises treating the mycobacterial cells with the test substance under conditions permitting the test substance to affect the mycobacterial cells.

In the disclosed methods, the mycobacterial cells are washed and seeded into a multi-well plate (e.g., a 96-well plate) at a predetermined number of cells per well. The number of mycobacterial cells per well can be modified as needed to optimize the assay. For example, the number of mycobacterial cells per well can be about 1×10⁴ colony forming units per well (cfu/well) to about 1×10⁷ cfu/well, or about 5×10⁴ cfu/well to about 5×10⁶ cfu/well, or about 5×10⁴ cfu/well to about 5×10⁵ cfu/well, but is not limited thereto. The test substance is added to the mycobacterial cells in the wells at varying concentrations and the mycobacterial cells are incubated with the test substance at a temperature of about 30° C. to about 37° C. or about 32° C. to about 37° C., for a time period of about 12 hours to about 72 hours, about 12 hours to about 60 hours, or about 12 hours to about 48 hours, or about 18 hours to about 72 hours, or about 18 hours to about 60 hours, or about 18 hours to about 48 hours, or about 24 hours to about 72 hours, or about 24 hours to about 60 hours, or about 24 hours to about 48 hours. In an aspect, the treated mycobacterial cells are incubated with the test substance at a temperature of about 37° C. for a time period of about 12 hours, about 18 hours, about 24 hours, about 36 hours, about 48 hours, about 60 hours, or about 72 hours. In an aspect, the test substance is incubated with the mycobacterial cells at a temperature of about 37° C., for a time period of about 24 hours to about 48 hours.

Following treatment with the test substance, the mycobacterial cells are contacted with the reporter mycobacteriophage. The contacting of the treated mycobacterial cells with the reporter mycobacteriophage comprises exposing the treated mycobacterial cells to the reporter mycobacteriophage under conditions permitting the reporter mycobacteriophage to infect the treated mycobacterial cells. Thus in an aspect, the contacting of the treated mycobacterial cells with the reporter mycobacteriophage comprises infecting the treated mycobacterial cells with the reporter mycobacteriophage. The number of reporter mycobacteriophage added to the treated cells can be modified as needed to optimize infection. For example, the ratio of the number of reporter mycobacteriophage to the number of mycobacterial cells may be about 1:1, or about 2:1, or about 5:1, or about 10:1, or about 20:1, but is not limited thereto. In an aspect, the number of reporter mycobacteriophage per well can be about 1×10⁴ plaque forming units per well (pfu/well) to about 1×10⁷ pfu/well, but is not limited thereto. The conditions facilitating infection of a viable target microbe with the reporter mycobacteriophage comprise those previously described herein. In an aspect, the sample is incubated with the reporter mycobacteriophage at a temperature of about 30° C. to about 37° C., for a time period of about 4 to 48 hours, or about 4 hours to about 24 hours, or about 4 hours to about 12 hours.

In an aspect, the contacting further comprises detecting and/or measuring the expression of the fusion protein in the treated mycobacterial cells contacted with the reporter mycobacteriophage. As discussed previously, the reporter mycobacteriophage infects and induces the expression of the fusion protein in a viable mycobacterial cell. Thus, when a viable treated mycobacterial cell is contacted with and infected by the reporter mycobacteriophage the promoter is activated, the fusion protein is expressed, and fluorescent light is emitted from the cell. The presence or absence of viable treated mycobacterial cells is thus determined by detecting the expression of the fusion protein in the treated mycobacterial cells contacted with the reporter mycobacteriophage. In an aspect, expression of the fusion protein indicates that a viable treated mycobacterial cell is present. In contrast, the absence of detectable signal indicates that there are no viable mycobacterial cells present, or that the number present is below a threshold of detection. In an aspect, decreased expression of the fusion protein relative to mycobacterial cells which have not been treated with the test substance, indicates that the test substance has decreased the viability of the mycobacterial cells (i.e., at least a portion, or all of the mycobacterial cells have been killed).

In the methods disclosed herein, detecting expression of the fusion protein in the treated mycobacterial cells contacted with the reporter mycobacteriophage comprises adding a substrate to the sample, reacting the substrate with fusion protein present in the sample, and measuring an amount of fluorescent light emitted from the sample. The adding of the substrate to the sample, the reacting of the substrate with the fusion protein, and the measuring of fluorescent light emitted from the sample, are encompassed by the methods previously described herein.

The amount of fluorescent light emitted from the sample can therefore be used to determine whether the test substance has, or has not, decreased the number of viable mycobacterial cells. The effectiveness of the test substance can be evaluated by calculating the percent inhibition. The percentage inhibition of the treated mycobacterial cells contacted with the reporter mycobacteriophage can be determined (calculated) by comparing an amount of the fusion protein detected in the treated mycobacterial cells with an amount of the fusion protein detected in a positive control. The positive control includes the same number of mycobacterial cells which are not contacted with the test substance. In other words, percent inhibition is determined by comparing the amount of the fusion protein expressed in the presence of the test substance and in the absence of the test substance, where a decrease in expression indicates that the mycobacterial cells are susceptible to the test substance. A standard curve based on known numbers of mycobacterial cells can also be used to correlate mycobacterial cell number with levels of fluorescence in order to more accurately quantify the number of viable cells. An antibiotic known to have antimycobacterial activity on the mycobacterial cells can be used as a separate control. Examples of such antibiotic include isoniazid, kanamycin, and rifampicin.

The minimum inhibitory concentration of the test substance can be determined by those of skill in the art based upon the above-described methods.

The present disclosure also provides a diagnostic method for detecting a drug-resistant mycobacterial cell in a sample, the method comprising:
providing a sample comprising the mycobacterial cell;
contacting the sample with the drug to treat the mycobacterial cell in the sample;
adding a reporter mycobacteriophage capable of infecting the mycobacterial cell to the sample, wherein the reporter mycobacteriophage comprises a heterologous nucleic acid comprising a promoter-reporter construct encoding a promotor operably linked to a nucleotide sequence encoding a fusion protein, wherein the fusion protein comprises a luciferase protein linked to a fluorescent protein; and
detecting expression of the fusion protein in the sample, wherein expression of the fusion protein indicates that the mycobacterial cell is resistant to the drug.

In an aspect, expression of the fusion protein indicates that the treated mycobacterial cell is not susceptible to the drug. In an aspect, the absence of fusion protein expression indicates that the treated Mycobacterium strain is substantially susceptible to the drug.

In an aspect, the sample is a biological sample from a mammalian subject, and specifically, a human subject. The biological sample can comprise sputum, blood, throat swab, genital swab, urethral swab, body fluids (CSF and others) or a combination thereof. In an aspect, the Mycobacterium strain is M. tuberculosis. In an aspect, the M. tuberculosis is a multidrug resistant tuberculosis (MDR-TB) strain, an extensively drug resistant tuberculosis (XDR-TB) strain, or a combination thereof. In an aspect, the M. tuberculosis strain is resistant to, or is suspected of being resistant to kanamycin, isoniazid, rifampicin, or a combination thereof. The biological sample can be tested directly or subjected to preliminary processing prior to testing.

Throughout the present disclosure, the Mycobacterium strain can comprise M. tuberculosis, M. bovis, M. smegmatis, M. bovis-BCG, M. avium, M. phlei, M. fortuitum, M. lufu, M. paratuberculosis, M. habana, M. scrofulaceum, M. intracellularae, or a combination thereof, or any other known mycobacteria, including those described hereinabove.

Throughout the present disclosure, the methods disclosed herein involving subjects can be used with any mammalian subject. In an aspect, the subject is a human.

The methods disclosed herein are highly sensitive and can be used to detect the presence of very low numbers of viable mycobacterial cells present in a given sample. In particular, the methods can be used to detect less than or equal to about 500, less than or equal to about 100, less than or equal to about 75, less than or equal to about 50, or less than or equal to about 40 viable mycobacterial cells.

This disclosure is further illustrated by the following examples, which are non-limiting.

### Examples

### Construction of Reporter Mycobacteriophage

Cosmids containing a fusion protein reporter cassette under control of an L5 promoter (SEQ ID NO:1), were constructed in a pYUB vector. The reporter cassettes included a nucleic acid sequence of a luciferase protein connected to a nucleic acid sequence of a fluorescent protein. A cosmid carrying a mNeon_green-nLuc cassette (Addgene), a cosmid carrying mNeon_green-nLuc cassette with Mycobacterium OriM (SEQ ID NO:2) origin of replication, a cosmid carrying mTurquoise-nLuc cassette, a cosmid carrying mNeon_green-nLuc-mTurquoise cassette, a cosmid carrying mNeon_green-nLuc-mNeon_green cassette, a cosmid carrying mTurquoise-nLuc-mTurquoise cassette, and a cosmid carrying CyOFP-teLuc-CyOFP cassette (Antares2; Addgene), were each constructed.

The cosmids were digested with PacI, ligated into a PacI-digested conditionally replicating (temperature sensitive) TM4 mycobacteriophage backbone (phAE159), and subjected to in vitro packaging to construct shuttle phasmids. The phAE159 mycobacteriophage injects its DNA into the mycobacterial cells at a temperature of 37° C. but propagates as a phage at 30° C. The maps of the shuttle phasmids carrying the mNeon_green-nLuc cassette (phAE1142) and the Antares2_nLuc cassette (phAE1158) are shown in FIGS. 1A and 1B, respectively. The shuttle phasmids were electroporated into host strain M. smegmatis (strain mc²155) following standard protocols for electroporation of Mycobacterium. Individual plaques were picked and propagated in mc²155 as a host strain, and high titer mycobacteriophage lysates were prepared.

Measurement of Antibiotic Susceptibility in M. tuberculosis

The ability to detect the susceptibility of M. tuberculosis to an antibiotic using the reporter mycobacteriophage was evaluated.

M. tuberculosis strains mc²6230, mc²8245, and mc²8247 were used. M. tuberculosis strain mc²6230 is a drug-sensitive ΔRDI ΔpanCD mutant, M. tuberculosis strain mc²8245 (ΔpanCD ΔleuCD ΔargB) is resistant to isoniazid (INH), and M. tuberculosis strain mc²8247 (ΔpanCD ΔleuCD ΔargB) is resistant to rifampicin (RIF). The reporter mycobacteriophage detection assay includes a positive signal control sample (Mycobacterial cells infected with mycobacteriophage, without antibiotic) and a background noise control (no bacteria; mycobacteriophage alone).

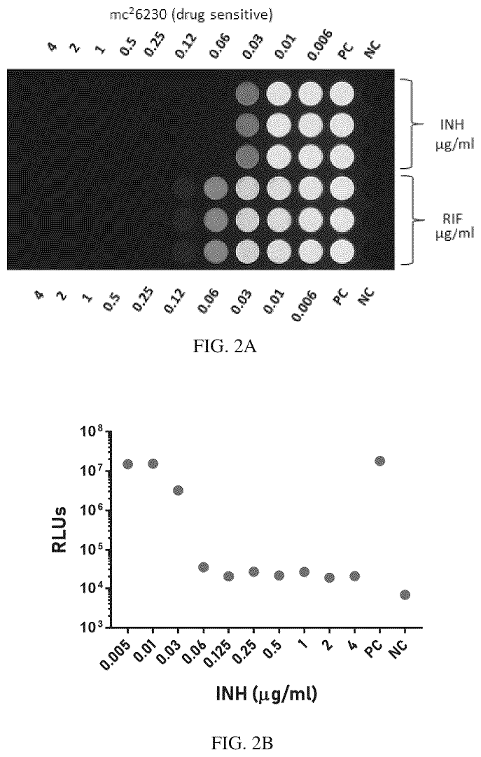

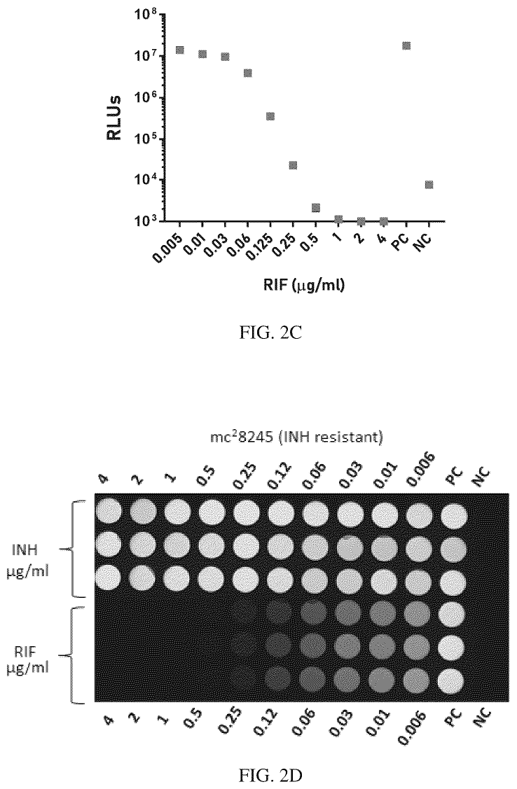

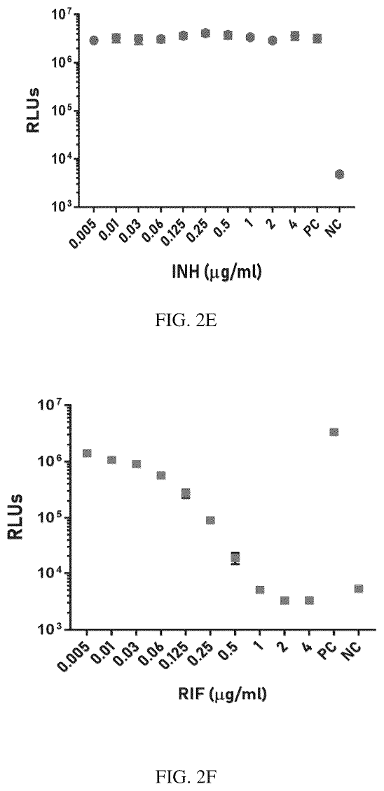

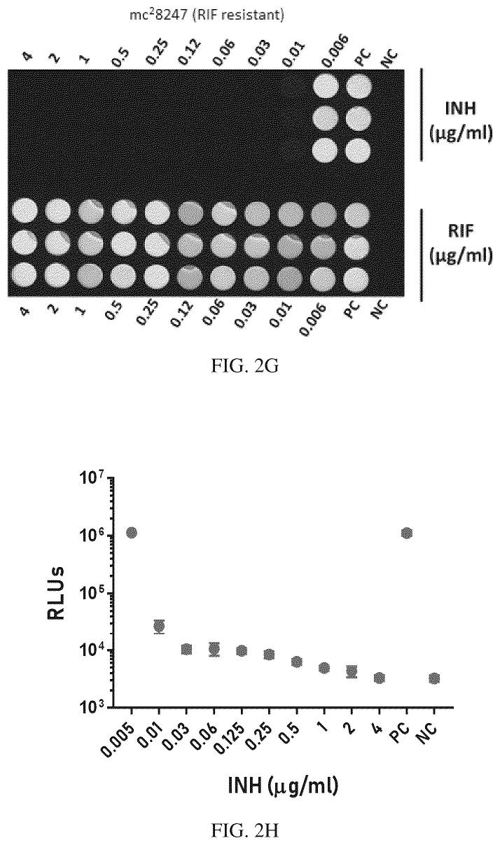

M. tuberculosis cells were washed and seeded into wells of a 96-well opaque plate containing 7H9 media at a density of 10⁵ cfu per well, and the antibiotics isoniazid and rifampicin were added in concentrations ranging from 0.006 μg/ml to 4 μg/ml. The plates were then incubated at 37° C. for 48 h, followed by infection with 10⁵ pfu/well of phAE1142 phage, and additional incubation at 37° C. for 24 h. Furimazine substrate was added to the wells, and the plate or plates were placed in a device configured to take photographic images of luciferase cell growth (Bronx Box 3.0; constructed in house), and the images were captured using a 2 min exposure. The plate images results are shown in FIG. 2A (strain mc²6230), FIG. 2D (strain mc²8245), and FIG. 2G (strain mc²8247). The dose response curves are shown in FIGS. 2B-2C (strain mc²6230), in FIGS. 2E-2F (strain mc²8245), and FIGS. 2H-2I (strain mc²8247).

Prophetic Example: The above-described assay can be compared to commercially available tests such as the Line Probe Assay (LPA), the Xpert MTB/RIF test (GeneExpert, Cepheid, USA), and culture methods. The LPA (e.g., INNO-LiPA RIF TB, Innogenetics, Belgium; Genotype MTBDRplus, Hain Life-Science, Germany) is based on reverse hybridization of DNA, and has a turnaround time of 2 to 3 days. The Xpert MTB/RIF test is based on real time PCR and has a turnaround time of 3 hours or less.

Detection of M. tuberculosis

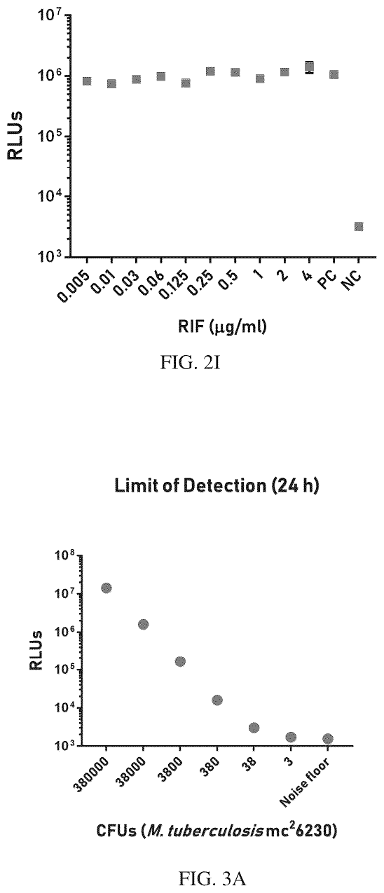

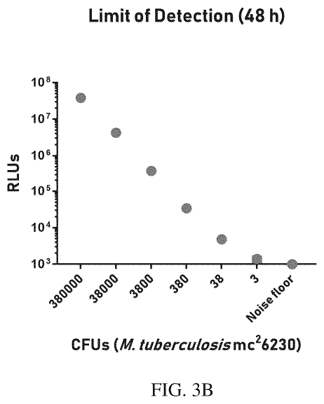

The sensitivity of the reporter mycobacteriophage to detect viable mycobacterial cells was also investigated. M. tuberculosis strain mc²6230 cells were washed and re-suspended in rich growth medium without detergent and inoculated in 10-fold serial dilutions, into wells of 96-well microtiter plates. phAE1142 phage (10⁶ pfu/well) were added immediately following transition of the cells to detergent-free medium. The plates were incubated for 24 h or 48 h at 37° C. followed by addition of furimazine to the wells. The results are shown in FIGS. 3A and 3B. In both cases, the cellular limit of detection is less than or equal to 100 viable Mycobacterium in a sample.

### DISCUSSION

Reporter mycobacteriophages including firefly luciferase (Jacobs, et al., Science (1993), 260(5109):819-822) and green fluorescent protein (Jain et al, J. Clin. Microbiol. (2012), 50(4): 1362-1369) have been developed to M. tuberculosis detection. A reporter mycobacteriophage for detection of M. tuberculosis drug persisters, which is a subpopulation of cells which remain metabolically inactive but viable in the presence of a drug, has also been developed (Jain et al, mBio (2016), 7(5); e01023-16; doi: 10.1128/mBio.01023-16). This is the first time that the disclosed enhanced ultra-sensitive nanoluciferase fusion proteins have been used in a reporter mycobacteriophage to develop a point of care diagnostic test. The reporter mycobacteriophage of the present disclosure is the most powerful phage generated to date with respect to signal intensity and detection sensitivity. The disclosed reporter mycobacteriophage is sensitive enough to detect as few as 100 mycobacteria cells and allows for rapid high throughput detection of M. tuberculosis in clinical samples. Further, the reporter mycobacteriophages do not require labor-intensive instruments, expertise, or expensive reagents making their implementation widely feasible and relatively inexpensive. Accordingly, the reporter mycobacteriophages of the present disclosure are superior to other systems for detecting, measuring, and quantifying viable M. tuberculosis.

The compositions, methods, and articles can alternatively comprise, consist of, or consist essentially of, any appropriate materials, steps, or components herein disclosed. The compositions, methods, and articles can additionally, or alternatively, be formulated so as to be devoid, or substantially free, of any materials (or species), steps, or components, that are otherwise not necessary to the achievement of the function or objectives of the compositions, methods, and articles.

All ranges disclosed herein are inclusive of the endpoints, and the endpoints are independently combinable with each other (e.g., ranges of “up to 25 wt. %, or, more specifically, 5 wt. % to 20 wt. %”, is inclusive of the endpoints and all intermediate values of the ranges of “5 wt. % to 25 wt. %,” etc.). “About” as used herein is inclusive of the stated value and means within an acceptable range of deviation for the particular value as determined by one of ordinary skill in the art, considering the measurement in question and the error associated with measurement of the particular quantity (i.e., the limitations of the measurement system). For example, “about” can mean within one or more standard deviations, or within ±20%, 10% or 5% of the stated value.

“Combinations” is inclusive of blends, mixtures, alloys, reaction products, and the like. The terms “first,” “second,” and the like, do not denote any order, quantity, or importance, but rather are used to distinguish one element from another. The terms “a” and “an” and “the” do not denote a limitation of quantity and are to be construed to cover both the singular and the plural, unless otherwise indicated herein or clearly contradicted by context. “Or” means “and/or” unless clearly stated otherwise. Reference throughout the specification to “some embodiments”, “an embodiment”, and so forth, means that a particular element described in connection with the embodiment is included in at least one embodiment described herein, and may or may not be present in other embodiments. In addition, it is to be understood that the described elements may be combined in any suitable manner in the various embodiments. A “combination thereof” is open and includes any combination comprising at least one of the listed components or properties optionally together with a like or equivalent component or property not listed.

Unless defined otherwise, technical and scientific terms used herein have the same meaning as is commonly understood by one of skill in the art to which this application belongs. All cited patents, patent applications, and other references are incorporated herein by reference in their entirety. However, if a term in the present application contradicts or conflicts with a term in the incorporated reference, the term from the present application takes precedence over the conflicting term from the incorporated reference.

While embodiments have been described, alternatives, modifications, variations, improvements, and substantial equivalents that are or may be presently unforeseen may arise to applicants or others skilled in the art. Accordingly, the appended claims as filed and as they may be amended are intended to embrace all such alternatives, modifications variations, improvements, and substantial equivalents.

1. A reporter mycobacteriophage comprising a heterologous nucleic acid comprising a promoter-reporter construct encoding a promotor operably linked to a nucleotide sequence encoding a fusion protein, wherein the fusion protein comprises a luciferase protein linked to a fluorescent protein.

2. (canceled)

3. The reporter mycobacteriophage of claim 1, wherein the luciferase protein comprises nLuc, teLuc, yeLuc, LumiLuc, an amino acid variant thereof, or a combination thereof.

4. The reporter mycobacteriophage of claim 1, wherein the fluorescent protein comprises mNeonGreen, moxNeonGreen, mTourquoise, mTourquoise2, cyan-excitable orange fluorescent protein (CyOFP), cyan-excitable red fluorescent protein (CyRFP), monomeric cyan-excitable red fluorescent protein (mCyRFP1), tdTomato, mCherry, mApple, mCardinal, mMaroon, mScarlett, mWassabi, an amino acid variant thereof, or a combination thereof.

5. The reporter mycobacteriophage of claim 1, wherein a first fluorescent protein is connected to the N-terminus of the luciferase protein and a second fluorescent protein is connected to the C-terminus of the luciferase protein.

6. The reporter mycobacteriophage of claim 1, wherein the fusion protein has a nucleic acid sequence which is at least 90% identical to SEQ ID NO: 3, SEQ ID NO: 5, SEQ ID NO:7, SEQ ID NO:9, SEQ ID NO: 11, or SEQ ID NO: 13.

7. A composition comprising the reporter mycobacteriophage of claim 1.

8. A method for detecting a viable mycobacterial cell in a sample, the method comprising: contacting the sample with the reporter mycobacteriophage of claim 1, wherein the reporter mycobacteriophage is capable of infecting the mycobacterial cell, and detecting expression of the fusion protein in the sample, wherein expression of the fusion protein indicates that the viable mycobacterial cell is present in the sample.

9. The assay of claim 8, wherein the mycobacterial cell comprises Mycobacterium tuberculosis, Mycobacterium bovis, Mycobacterium smegmatis, Mycobacterium bovis-BCG, Mycobacterium avium, Mycobacterium phlei, Mycobacterium fortuitum, Mycobacterium lufu, Mycobacterium paratuberculosis, Mycobacterium habana, Mycobacterium scrofulaceum, Mycobacterium intracellularae, or a combination thereof.

10. The assay of claim 8, wherein the mycobacterial cell comprises Mycobacterium tuberculosis.

11. The assay of claim 8, wherein the contacting comprises infecting the mycobacterial cell with the reporter mycobacteriophage and inducing expression of the fusion protein in the mycobacterial cell.

12. The assay of claim 8, wherein the detecting comprises adding a substrate to the sample, reacting the substrate with fusion protein present in the sample, and measuring an amount of fluorescent light emitted from the sample.

13. A kit for detection of a mycobacterial cell in a sample, comprising: a reporter mycobacteriophage capable of infecting the mycobacterial cell; a substrate for detecting the fusion protein; and instructions for performing the method of claim 8, wherein the reporter mycobacteriophage comprises a heterologous nucleic acid comprising a promoter-reporter construct encoding a promotor operably linked to a nucleotide sequence encoding a fusion protein, wherein the fusion protein comprises a luciferase protein linked to a fluorescent protein.

14. A method for screening a test substance in vitro for antimycobacterial activity, the method comprising: contacting mycobacterial cells with the test substance to provide treated mycobacterial cells; contacting the treated mycobacterial cells with the reporter mycobacteriophage of claim 1, wherein the reporter mycobacteriophage is capable of infecting the mycobacterial cells, detecting expression of the fusion protein in the treated mycobacterial cells contacted with the reporter mycobacteriophage, wherein expression of the fusion protein indicates the presence of a viable mycobacterial cell; and determining a percentage inhibition of the treated mycobacterial cells contacted with the reporter mycobacteriophage.

15. The method of claim 14, wherein the mycobacterial cells comprise Mycobacterium tuberculosis, Mycobacterium bovis, Mycobacterium smegmatis, Mycobacterium bovis-BCG, Mycobacterium avium, Mycobacterium phlei, Mycobacterium fortuitum, Mycobacterium lufu, Mycobacterium paratuberculosis, Mycobacterium habana, Mycobacterium scrofulaceum, Mycobacterium intracellularae, or a combination thereof.

16. The method of claim 14, wherein the contacting of the treated mycobacterial cells with a reporter mycobacteriophage comprises infecting the treated mycobacterial cells with the reporter mycobacteriophage and inducing expression of the fusion protein in the target microbe.

17. The method of claim 14, wherein the detecting comprises adding a substrate to the treated mycobacterial cells contacted with the reporter mycobacteriophage and measuring an amount of fluorescent light emitted.

18. A method for detecting presence of a drug-resistant mycobacterial cell in a sample, the method comprising: providing a sample comprising the mycobacterial cell; contacting the sample with the drug to treat the mycobacterial cell in the sample; adding theft reporter mycobacteriophage of claim 1, wherein the reporter mycobacteriophage is capable of infecting the mycobacterial cell to the sample; and detecting expression of the fusion protein in the sample, wherein expression of the fusion protein indicates that the treated mycobacterial cell is resistant to the drug.

19. The method of claim 18, wherein the sample is a biological sample from a mammalian subject, and wherein the biological sample comprises sputum, blood, throat swab, genital swab, urethral swab, or a combination thereof.

20. The method of claim 18, wherein the mycobacterial cell comprises Mycobacterium tuberculosis, Mycobacterium tuberculosis, Mycobacterium bovis, Mycobacterium smegmatis, Mycobacterium bovis-BCG, Mycobacterium avium, Mycobacterium phlei, Mycobacterium fortuitum, Mycobacterium lufu, Mycobacterium paratuberculosis, Mycobacterium habana, Mycobacterium scrofulaceum, Mycobacterium intracellularae, or a combination thereof.

21. The method of claim 19, wherein the mycobacterial cell comprises Mycobacterium tuberculosis, Mycobacterium tuberculosis, Mycobacterium bovis, Mycobacterium smegmatis, Mycobacterium bovis-BCG, Mycobacterium avium, Mycobacterium phlei, Mycobacterium fortuitum, Mycobacterium lufu, Mycobacterium paratuberculosis, Mycobacterium habana, Mycobacterium scrofulaceum, Mycobacterium intracellularae, or a combination thereof.
